(19) United States
(12) Patent Application Publication
(10) Pub. No.: US 2020/0229808 A1
(43) Pub. Date: Jul. 23, 2020

(54) RETRACTOR AND MODULAR EXTENSIONS AND INSTRUMENTS

(71) Applicant: Javier GARCIA-BENGOCHEA, Jacksonville, FL (US)

(72) Inventors: Javier GARCIA-BENGOCHEA, Jacksonville, FL (US); Marc VON AMSBERG, Waxhaw, NC (US)

(73) Assignee: Javier GARCIA-BENGOCHEA, Jacksonville, FL (US)

(21) Appl. No.: 16/487,780

(22) PCT Filed: Feb. 21, 2018
(86) PCT No.: PCT/US2018/019029

§ 371 Date: Aug. 21, 2019

Related U.S. Application Data
(60) Provisional application No. 62/461,709, filed on Feb. 21, 2017.

(51) Int. Cl. A61B 17/02

(52) U.S. Cl. CPC A61B 17/0218; A61B 1/32; A61B 17/0206

(57) ABSTRACT

A surgical retractor system including a retractor tube, one or more modular independent retractor extension clips removably engageable with the retractor tube to deflect and minimize tissue creep, and a deployment instrument for deploying and recovering retractor extension clips.

### RELATED APPLICATIONS

This application is a United States National Stage Application filed under 35 U.S.C. 371 and claims priority to PCT Patent Application No. PCT/US2018/019029 filed on Feb. 21, 2018, which application claims the benefit under 35 U.S.C. § 119(e) to U.S. Provisional Application Ser. No. 62/461,709 filed Feb. 21, 2017, the entirety of which is incorporated herein by reference.

### FIELD

The present application describes various exemplary instruments, systems and surgical techniques for achieving access to and placement of implants at a site within the body, particularly the spine. More particularly, the present application describes instruments and a system useful for accessing the spine for one or more purposes of tissue manipulation, and placement of intervertebral implants to supplement or replace natural spinal discs.

### DESCRIPTION OF THE RELATED ART

This invention relates generally to the field of devices utilized in surgery to retract and retain tissue, organs or the like to provide the surgeon with access or an unobstructed pathway to an organ, bone, tissue or point in the body. Such devices are typically referred to generically as retractors. More particularly, the invention relates to retractors utilized with minimally invasive surgical techniques, wherein minimally sized openings are created in the body rather than relatively large incisions. Tubular devices known as cannula retractors, or spreading retractors having finger-like retractor extensions or blades, are inserted into the small opening and through or around the body tissues, muscles, tendons, ligaments, etc., thereby minimizing damage to the body. The surgeon then performs the necessary procedure through the retractor tube using specially designed tools and equipment.

A problem with known retractors used in minimally invasive surgery is that the distal end profile or configuration of the device is fixed. A typical tubular retractor has a circular or elliptical transverse cross-section with the distal end lying in the plane perpendicular or slanted relative to the longitudinal axis. Other tubular distractors may have non-planar ends of varying configurations, such as having a spatula-like extension. Spreading retractors likewise have varying end configurations. Because the distal end configurations of the devices are fixed, the devices often fail to prevent creep or herniation of tissue at or around the distal end of the device, and this tissue creep can interfere with the necessary physical or visual access. This is particularly true when the distal end of the retractor device is positioned adjacent or near a bone, such as a vertebra for example, that does not have a planar or smooth outer contour, or where the retractor device is disposed at a non-perpendicular angle to the bone. In these circumstances it often becomes necessary for the surgeon to cut away the interfering tissue, thereby creating additional tissue damage that needs to heal and increasing the possibility of detrimental results such as bleeding, increased pain, infection and the like.

Some retractors of the spreadable blade type provide shim members that are mounted in interior channels disposed in each of the blades. The shim can be extended beyond the distal end of the blade by sliding the shim relative to the blade. The shortcomings of this solution to the problem of tissue creep is that the location of the shims are determined by the location of the blades after they have been positioned and spread. Thus, the surgeon cannot address the problem of tissue creep that occurs between the blades.

### SUMMARY

In accordance with the disclosure, systems, instruments and techniques are provided for enhancing visualization through a retractor during a surgical procedure, particularly a spinal surgery.

In an exemplary embodiment, a surgical retractor system includes a surgical retractor having at least one slot positioned through a wall of the retractor and at a point between proximal and distal ends, an extension clip having a flared blade, and a mounting means for releasable engagement with the at least one slot of the surgical retractor. The mounting means includes one or more of a spring type fitting that is actuatable between open and compressed configurations, an interference fitting, a compression fitting, and a snap fitting. The system also includes a deployment instrument having an instrument body with a releasable actuation means for engaging the mounting means of the extension clip and for one of affixing or releasing the extension clip in relation to the surgical retractor slot. The actuation means is adapted to direct locking engagement of the extension clip mounting means with the surgical retractor, and includes one or more of a releasable gripper for grasping and compressing a spring type fitting, and a biasing means for effecting one or more of compression and snap fitting engagement and an actuator handle in operable communication with the releasable actuation means of the instrument body. The system is adapted for engagement of the extension clip with the surgical retractor body slot to mount the extension in a locked position for extension of the blade toward or outside of the distal end of the surgical retractor body. In use, when fixed to the surgical retractor, at least a portion of the extension blade is positioned to contact soft tissue adjacent the distal end of the surgical retractor and thereby minimize tissue creep into the surgical retractor. In some examples, the surgical retractor has an elongate body. In some specific examples, the surgical retractor has a generally cylindrical or ellipsoid elongate body defining a lumen between its proximal and distal ends and comprising at least one slot disposed through the elongate body toward the distal end.

In another exemplary embodiment, an extension clip for a surgical retractor is provided. The extension clip includes a body having at a proximal end a mounting means for releasable engagement with at least one slot of the surgical retractor, the mounting means having one or more of a spring type fitting that is actuatable between open and compressed configurations, an interference fitting, a compression fitting, and a snap fitting, and at a distal end a flared blade. The extension clip is adapted for engagement with the surgical retractor slot in a releasably locked configuration for positioning the blade toward or outside of a distal end of the surgical retractor. In use, when fixed to the surgical retractor, at least a portion of the extension blade is positioned to contact soft tissue adjacent the distal end of the surgical retractor and thereby minimize tissue creep into the surgical retractor.

In one example, the mounting means of the extension clip has a spring-type interference fitting that is adapted to be actuated between open and compressed configurations. According to such embodiment, in the compressed configuration, the mounting means can be passed through the surgical retractor slot, and in the open configuration the mounting means is retained in the surgical retractor slot by interference fitting. The extension clip mounting means includes opposing flexible tabs that are proximal to the distal blade, each tab having a hook that includes a pull out stop for interference with a wall of the surgical retractor when inserted in the slot. The tabs are separated from each other by an open aperture. Flexion of the tabs within the aperture moves the hooks toward each other in a compressed configuration, allowing for passage of the hooks through the slot, and release of the tabs returns the mounting means to an open configuration.

In another example, the mounting means of the extension clip includes a locking tab affixed by a tab arm and extending from a back side of the extension clip. The tab arm includes on its opposing sides locking divots. The arm of the locking tab is engageable with a surgical retractor slot that is variably dimensioned along a length between proximal and distal ends of the surgical retractor. In accordance with such embodiments, the variably dimensioned slot includes a distally oriented portion having a width dimension that is greater than a width dimension of a proximally oriented portion. Upon its passage into the distally oriented slot portion and proximal translation into the proximally oriented slot portion, the extension tab may be releasably locked to the surgical retractor, and is prevented from pull out by interference between the locking tab and the surgical retractor body.

In another example, the mounting means of the extension clip includes a locking tab affixed by a tab arm and extending from a back side of the extension clip, the tab arm having a cross sectional shape from distal to proximal that is conical or frustoconical. The arm of the locking tab is engageable with a surgical retractor slot that is variably dimensioned along a length between the proximal and distal ends of the surgical retractor. The variably dimensioned slot comprising a distally oriented portion and a proximally oriented portion, the distally oriented portion having a greater width dimension than a width dimension of the proximally oriented portion, the proximally oriented portion tapering from the distally oriented portion in a shape that is conical or frustoconical. Upon its passage into the distally oriented slot portion and proximal translation into the proximally oriented slot portion, the extension tab can be releasably locked to the surgical retractor, and is prevented from pull out by interference between the locking tab and the surgical retractor body.

In another example, the mounting means of the extension clip includes at least two vertically separated hooks directed proximally and extending from a back side of the extension clip, at least one hook being distally positioned and comprising a locking flange. The at least two vertically separated hooks are engagable, respectively, with at least one slots and the distal end of a surgical retractor. The locking flange is releasably locked to one of a slot and a distally positioned edge of the surgical retractor achieved by actuating snap fit locking. In some embodiments, the extension clip includes more than two vertically separated hooks, one or more of which hooks comprise a locking flange. According to the various embodiments, the extension clips are adapted to engage with a retractor that includes one slot or two or more vertically separated slots.

In another exemplary embodiment, a surgical retractor extension deployment instrument is provided. The instrument includes an instrument body with a releasable actuation means for engaging a mounting means of the extension clip and for one of affixing or releasing the extension clip in relation to at least one slot in the surgical retractor. The actuation means is adapted to direct locking engagement of the extension clip mounting means with the surgical retractor, and includes one or more of a releasable gripper for grasping and compressing a spring type fitting, and a biasing means for effecting one or more of interference, compression and snap fitting engagement. The instrument also includes an actuator handle in operable communication with the releasable actuation means of the instrument body.

In one example, the surgical retractor extension deployment instrument includes a releasable gripper comprising opposing grips that are engageable with the extension clip mounting means, and the handle includes a trigger that alternately directs the action of the opposing grips between open and closed configurations.

In another example, the deployment instrument includes scissor like handles at a proximal end, and oriented distally, opposing arms that include an insertion arm and a biasing arm. The handles and the insertion and biasing arms are joined, for example, by a hinge positioned therebetween. The biasing arm includes a distal biasing means that is adapted to be compressed against a surface that opposes a slot that is adapted to releasably receive an extension clip when the handles are one of squeezed and released. In some examples, the biasing means is a planar or curved surface that is adapted for either continuous contact with a retractor surface, or that is engageable with one or more slots, apertures or other features of the retractor. In an example, the biasing means on the biasing arm may be a torque plate or other planar or curved feature to enhance the contact surface area of the biasing arm. In some such embodiments, the torque plate is hinged or swiveled to vary its orientation as the instrument is moved.

In some examples, the instrument is adapted for use with a surgical retractor that has a generally cylindrical or ellipsoid elongate body defining a lumen between its proximal and distal ends and having at least one slot disposed through the elongate body toward the distal end. In some embodiments, the slot may be variably dimensioned along a length between the proximal and distal ends of the surgical retractor, and may include a distally oriented portion and a proximally oriented portion. According to such embodiments, the proximally oriented portion has at least a width dimension that is smaller than a width dimension of the distally oriented portion. In some specific embodiments, the proximally oriented portion includes on each of opposing internal side edges of the wall one or more nubs adapted for interfitting with locking divots on a part of an extension clip.

In some examples, the instrument is adapted for use with a surgical retractor comprising at least two vertically adjacent slots.

### BRIEF DESCRIPTION OF THE DRAWINGS

Features and advantages of the general inventive concepts will become apparent from the following description made with reference to the accompanying drawings, including drawings represented herein in the attached set of figures, of which the following is a brief description:

FIG. 1 shows three alternate views of a representative embodiment of a retractor and a first embodiment of an extension clip, according to the disclosure;

FIG. 2 shows three alternate views of a representative embodiment of a retractor and a second embodiment of an extension clip, according to the disclosure;

FIG. 3 shows three alternate views of a representative embodiment of a retractor and a third embodiment of an extension clip, according to the disclosure;

FIG. 4 shows three alternate views of a representative embodiment of a retractor and a fourth embodiment of an extension clip, according to the disclosure;

FIG. 5 shows alternate perspective, front, back, side, top and bottom views of a first embodiment of an extension clip, according to the disclosure;

FIG. 6 shows alternate perspective, front, back, side, top and bottom views of a second embodiment of an extension clip, according to the disclosure;

FIG. 7 shows alternate perspective, front, back, side, top and bottom views of a third embodiment of an extension clip, according to the disclosure;

FIG. 8 shows alternate perspective, front, back, side, top and bottom views of a fourth embodiment of an extension clip, according to the disclosure;

FIG. 9 shows alternate top and bottom views of a representative embodiment of a retractor and the embodiment of an extension clip having an angled tip as shown in FIG. 7, according to the disclosure;

FIG. 10 shows a perspective view of an embodiment of an extension deployment instrument according to the disclosure for placement and removal of an extension clip that is engageable with a retractor according to the embodiment shown in FIG. 1;

FIG. 11 shows an exploded perspective view of the extension deployment instrument shown in FIG. 10;

FIG. 12 shows an alternate view of the extension deployment instrument shown in

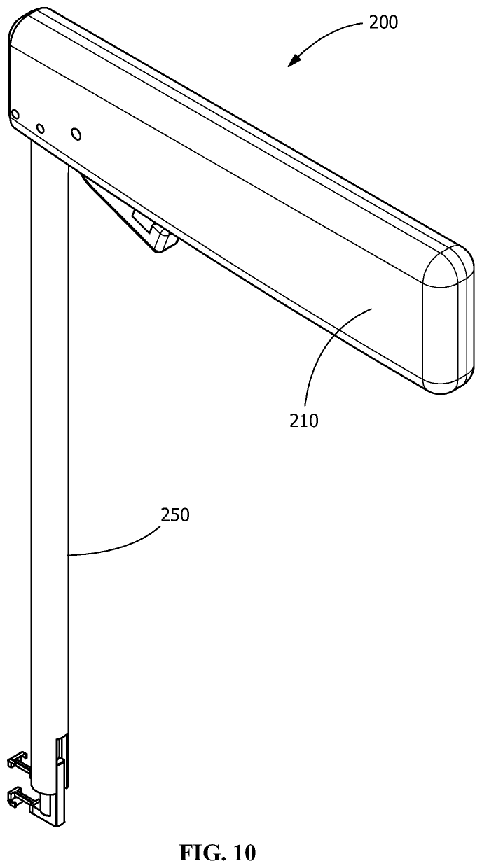

FIG. 10;

FIG. 13 shows, respectively from left to right, bottom and top views of the extension deployment instrument shown in FIG. 10;

FIG. 14 shows, respectively from left to right, back and front views of the extension deployment instrument shown in FIG. 10;

FIG. 15 shows an exploded side view of the extension deployment instrument shown in FIG. 10;

FIG. 16 shows, respectively from left to right, exploded bottom and top views of the extension deployment instrument shown in FIG. 10;

FIG. 17 shows, respectively from left to right, back and front views of the extension deployment instrument shown in FIG. 10;

FIG. 18 shows a detailed view of the distal engagement end of the deployment instrument;

FIG. 19 shows an alternate embodiment of a deployment instrument in alternate front and side views; and

FIG. 20 shows the embodiment of the deployment instrument shown in FIG. 19 engaged with an embodiment of an extension clip as shown in FIG. 7.

Reference Numeral Key:

| | | | |
|---|---|---|---|
| 100 | Retractor | 28 | Front |
| 102 | Lumen | 29 | Back |
| 104 | Slot | | |
| 106 | Proximal end | 30 | Extension Clip |
| 108 | Distal End | 31 | Mounting Means |
| 110 | Tubular Body | 32 | Locking Divot |
| | | 33 | Locking Tab |
| 114 | Variably dimensioned Slot | 34 | Tab Arm |
| 115 | Insertion slot | 35 | Tool Engagement Aperture |
| 116 | Locking slot | 36 | Angled Distal Blade |
| 117 | Nubs | 37 | Proximal End |
| | | 38 | Front |
| 10 | Extension Clip | 39 | Back |
| 11 | Mounting Means | | |
| 12 | Flexible Tab | 40 | Extension Clip |
| 13 | Hook | 41 | Mounting Means |
| 14 | Hook Arm | 42 | Slot Hook |
| 15 | Stub/Pull out stop | 43 | Distal Hook |
| 16 | Distal Blade | 44 | Locking Flange |
| 17 | Proximal End | 45 | Tool Engagement Aperture |
| 18 | Front | 46 | Distal Blade |
| 19 | Back | 47 | Proximal End |
| | | 48 | Front |
| 20 | Extension Clip | 49 | Back |
| 21 | Mounting Means | | |
| 22 | Locking Divot | 200 | Deployment Instrument |
| 23 | Locking Tab | 210 | Actuator Handle |
| 24 | Tab Arm | | |
| 25 | Tool Engagement Aperture | 220 | Handle |
| 26 | Distal Blade | 230 | Trigger |
| 27 | Proximal End | 235 | Flange |
| 250 | Actuator Body | 290 | Releasable gripper |
| 260 | Body Housing | 292 | Opposing clamp |
| 262 | Reciprocating Grip Retainer Guide | 300 | Inserter Instrument |
| 264 | Fixed Opposing Grip | 310 | Handles |
| 265 | Trigger guide | 312 | Insertion Arm |
| | | 314 | Insertion Retainer |
| 280 | Reciprocating Grip Body | 315 | Hinge |
| 282 | Reciprocating Grip Retainer | 318 | Biasing Arm |
| 284 | Reciprocating Opposing Grip | 319 | Biasing Arm distal biasing means |
| 285 | Trigger mount | 320 | Pivoting counter torque plate |
| 286 | Spring | 321 | Plate hinge |

### DESCRIPTION

The invention addresses, among other issues, tissue creep, particularly with respect to retractors used in minimally invasive spinal surgical procedures, among others, by providing a retractor device for minimally invasive surgery that incorporates blade-like extension clips that may be selectively attached within or adjacent or to the end of a tubular shaped retractor, and also are suitable for attachment to other instruments that would benefit from extension clips that can contact and retract tissue. The extension clips may be mounted to the retractor at any point about or adjacent the distal end utilizing various mechanical joining techniques, and the extension clips may vary in shape, size, thickness and other characteristics. The extension clips may be pre-formed with a fixed curvature to match the interior curvature of the retractor, or they may be composed of a flexible material such that they adapt to the interior curvature of the retractor upon affixation, or both. The extension clips may be provided with teeth, apertures, slits or the like to better prevent tissue creep.

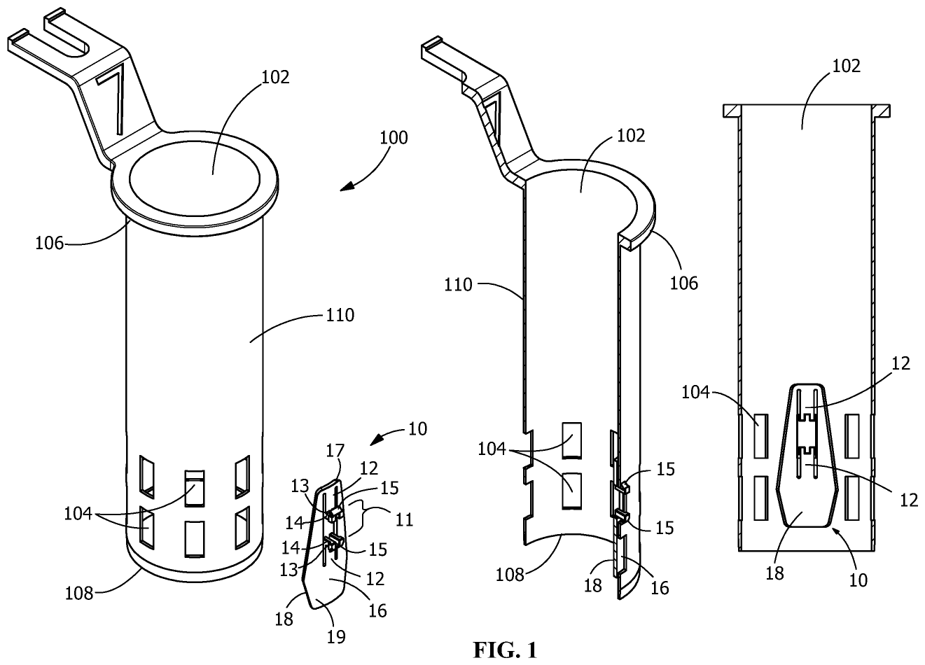

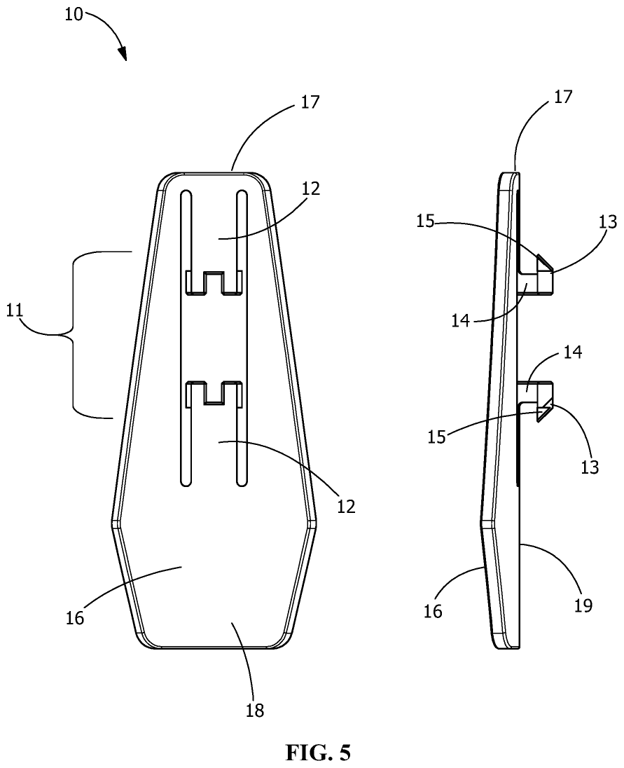

Referring now to FIG. 1, a representative embodiment of a surgical retractor system is shown, which includes (i) a retractor 100 having an elongated tubular body 110, a proximal end 106 and a distal end 108 and a lumen 102 defined therebetween through the tubular body 110, and at least one slot 104 positioned on the body at a point between a proximal and distal end; and, (ii) an extension clip 10 having a distal blade 16, and a mounting means 11 for releasable engagement with the at least one slot 104 of the retractor 100. Referring now to FIG. 5, the extension clip 10 as shown in FIG. 1 is shown in alternate views. The extension clip 10 includes at its proximal end 17 a mounting means 11 and a distal blade 16, and opposing front 18 and back 19 sides. As depicted, the mounting means 11 includes opposing flexible tabs 12 within the extension clip 10, the flexible tabs 12 separated from each other by an aperture, each flexible tab 12 having at its end within the aperture a hook 13. As depicted, the hook 13 includes a hook arm 14 and a pull out stop 15 that engages with the tubular body 110 when inserted through the slot 104 to provide an interference fit. The opposing hooks 13 can be actuated by flexion of the flexible tabs 12 to move the stubs toward each other in a compressed configuration and when released, return to an open configuration, the actuation allowing for insertion within a slot 104 on the retractor 100 whereby the pull out stops 15 of the opposing hooks 13 engage with the tubular body 110 of the retractor 100 preventing pull out until the opposing hooks 13 are again actuated by compression for removal from the slot 104.

As depicted, the extension clip 10 is adapted such that the front 18 side is to be oriented away from a contact surface of the retractor. With regard to the specific embodiment shown in FIG. 1 and FIG. 5, the front 18 side is oriented toward the lumen 102 of the retractor 100, and the back side 19 is adapted to contact the retractor 100. In some embodiments, the extension clip 10 has an overall flat planar shape. In other embodiments, as depicted in the drawings, the extension clip 10 has a curvature around its long axis from the proximal end 17 to the distal blade 16. In some embodiments the distal blade 16 is straight along the long axis, as depicted in FIG. 5, while in other embodiments, the distal blade 16 is deflected from the elongate axis, the deflection toward the back 19 side. It will be appreciated that the angulation of deflection may be varied from between 0 to 90 degrees off the long axis.

The system is adapted for engagement of the retractor extension clip 10 in a fixed, non-moving, position for extension of the distal blade 16 toward or outside of the distal end 108 of the retractor 100, whereby when at least a portion of the distal blade 16 extends outside the distal end 108 of the retractor 100, it is positioned to contact soft tissue and thereby minimizes or precludes tissue creep into the distal end 108 of the retractor 100. The inventive design provides for fixed and releasable engagement of an extension clip 10 with a retractor 100 such that the forces that would be directed on the extension clip 10 will tend to retain the fixation of the clip 10 to the retractor 100 rather than to urge it out of engagement. Thus, in the various embodiments, the extension clips 10 are retained on the retractor 100 either by spring type retention of a mounting means within an aperture such slot on the retractor, or by one of frictional/compressive or snap-fit engagement wherein the forces effecting engagement are directed along an axis that is parallel to the long axis of the retractor 100. As described below, the primary direction of force against the extension clips 10 would be expected from the sides and the bottom, which forces tend to reinforce the fixation of the extension clip 10.

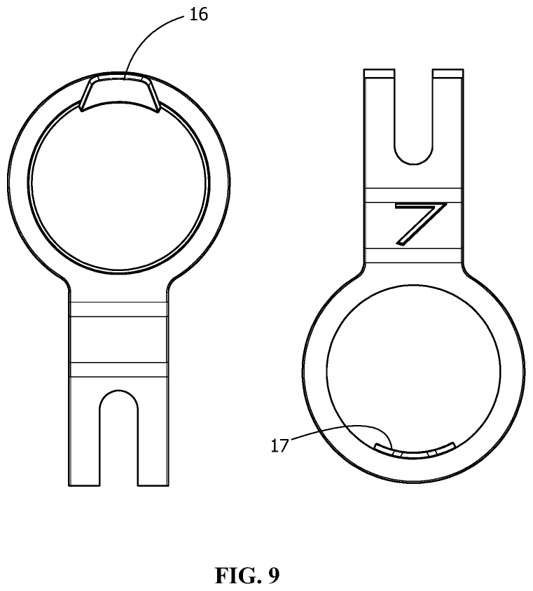

Referring again to the drawings in FIG. 1-10, in some embodiments, the tubular body 110 of the retractor 100 is adapted with a circumferential ring of slots allowing flexibility in the position of placement of the extension and allowing for multiple extension clips 10 to be positioned around the circumference of the retractor 100. It will be appreciated that the shape of the slots 104 may be varied, as well as their distribution and orientation around the circumference, as well as their proximal to distal positioning. Thus, in some embodiments, the slots 104 may be positioned at any location between the proximal 106 and distal 108 ends. In some embodiments, only one slot 104 may be included. And in other embodiments, two or more circumferential rows of slots 104 may be present. Thus, it will be appreciated that in various embodiments, the tubes may be adapted with slots 104 at more than one position along the length from the proximal 106 to distal 108 ends of the tubes, and that the shapes, sizes, number and distribution of the slots 104 may vary to accommodate varying sized extension clips 10 and to support instruments that may be affixed to an extension clip 10 along all or a portion of the length of the tubular body 110.

Of course, it will be appreciated that the tubular body 110 that is depicted in the drawings is merely representative, and other similar retractors 100 may be used in its place, for instance those that are formed of a unitary tubular or other shaped body, and those that may be assembled by the arrangement of an array of two or more panels, paddles or extension clips 10 to form a port or access to a surgical site. Thus, while the depicted embodiments of the inventive devices in this disclosure are shown in the context of a tubular retractor, it will be understood that the inventive devices may be used with other retractor devices as well as curved and planar retractors and similar instruments that are adapted with one or more slots for engagement with the inventive extension devices and suitable for insertion into a surgical space to retract tissue.

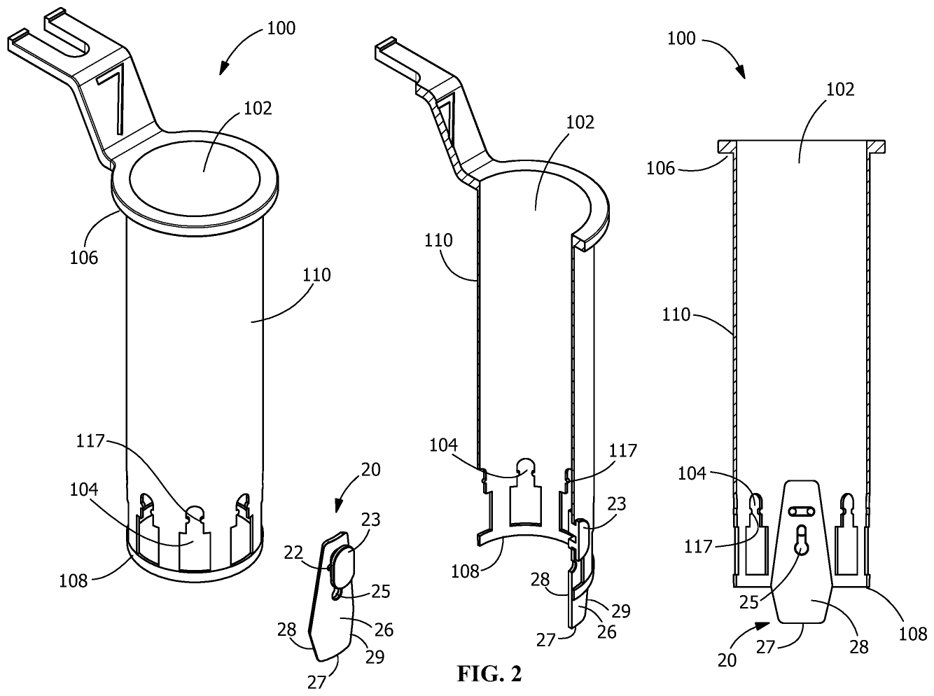

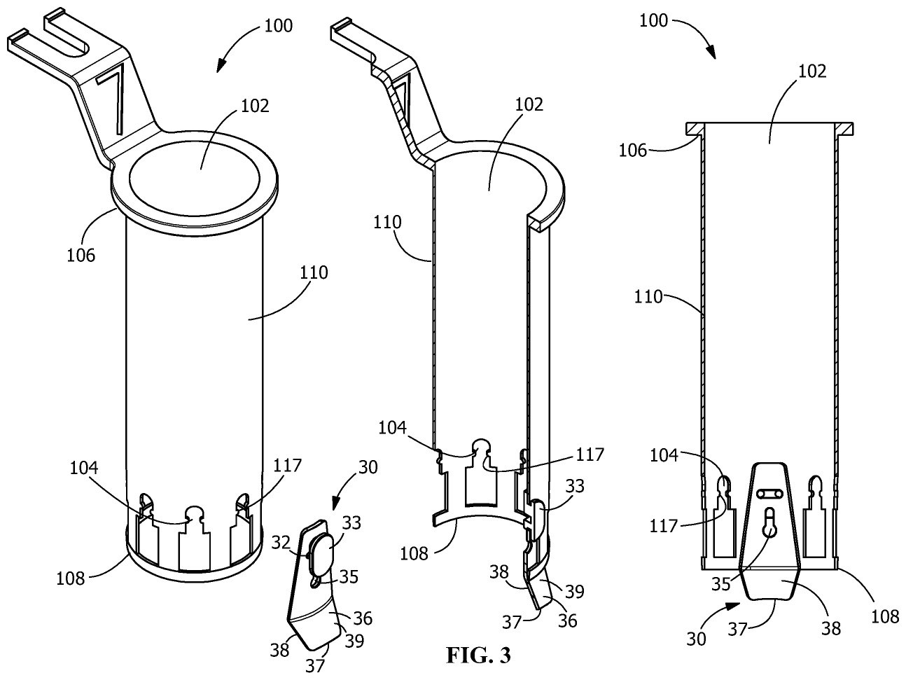

Alternate embodiments of extension clips are shown in each of FIGS. 2 and 5, FIGS. 3 and 7, and FIGS. 4 and 8. Referring now to FIG. 2 and FIG. 5, and FIG. 3 and FIG. 7, each of the depicted extension clips 20, 30 is adapted with a mounting means 21, 31 adjacent the proximal end 27, 37. The mounting means 21, 31 includes a locking tab 23, 33 affixed by a tab arm 24, 34. Each tab arm 24, 34 having a locking divot 22, 32 on each of opposing sides of the tab arm 24, 34. Each of the depicted extension clips 20, 30 is further adapted with at least one tool engagement aperture 25, 35, and a distal blade 26, 36, the extension clip 20, 30 being defined by front 28, 38 and back 29, 39 sides. As depicted, the extension clips 20, 30 are adapted such that the front 28, 38 sides are to be oriented away from a contact surface of the retractor.

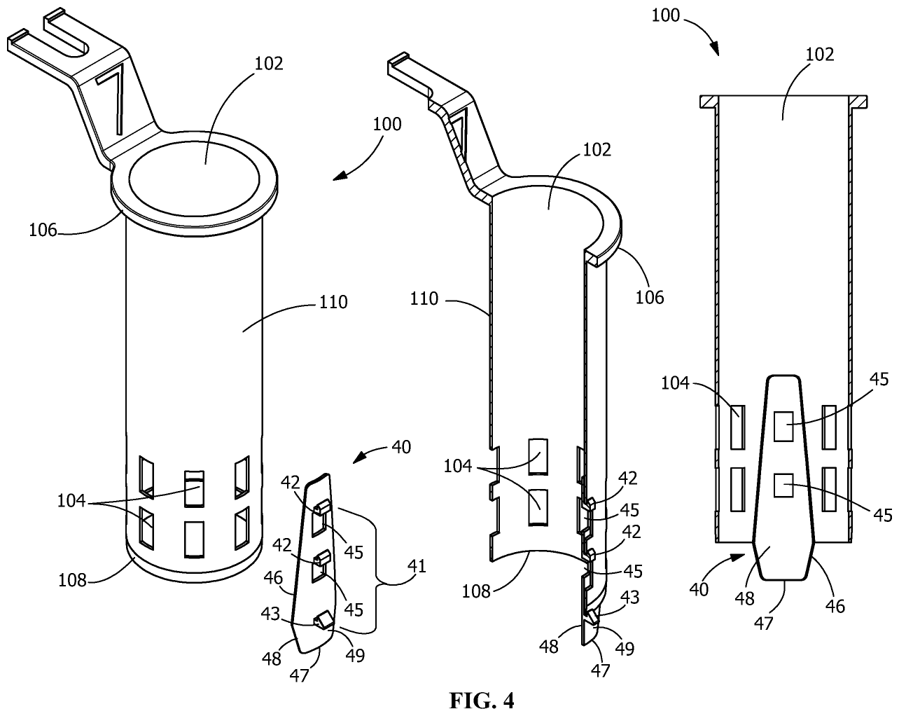

Yet another embodiment of an extension clip is shown in each of FIG. 4 and FIG. 8. The extension clip 40, as shown in FIG. 8, may be releasably engageable by the snap fit locking engagement at the distal end of the retractor, wherein the retractor includes at least two vertically adjacent slots 104. The extension clip 40 includes adjacent its proximal end 47 a mounting means 41 that includes three vertically separated hooks, two hooks 42, 43 being proximal to a third hook that comprises a distal locking flange 44, and at least one tool engagement aperture 45, a distal blade 46, and front and back sides 48, 49. It will be appreciated that in other embodiments, the clip may include more than three hooks and in other embodiments, it may include as few as two hooks. In accordance with the various embodiments, at least the distal most hook comprises a distal locking flange that is adapted for snap fitting with one of the end of or a slot of a retractor, the retractor being either planar or tubular. In some embodiments, more than one hook comprises a distal flange.

With regard to the specific embodiments shown in FIG. 2 and FIG. 3, the front 28, 38 sides are oriented toward the lumen 102 of the retractor 100. The back sides 29, 39 are adapted to contact the retractor. In some embodiments, the extension clips 20, 30 have an overall flat planar shape. In other embodiments, as depicted in the drawings, the extension clips 20, 30 have a curvature around their long axis from the proximal end 27, 37 to the distal blade 26, 36. Referring now to FIG. 3 and FIG. 7, the depicted embodiment has a distal blade that is deflected from the elongate axis, the deflection toward the back 39 side. It will be appreciated that the angulation of deflection may be varied from between 0 to 90 degrees off the long axis.

Referring now to FIG. 2 and FIG. 3, an alternate embodiment of a retractor is featured, the retractor adapted with variably dimensioned slot 114 for receiving the extension clip embodiments shown in FIGS. 2 and 6 and 3 and 7. Referring now to FIG. 3, the extension clip 30 may be releasably engageable within variably dimensioned slot 114, wherein a locking tab 33 of the extension clip 30 may be inserted into the variably dimensioned slot 114 at its wider insertion slot 115, aided by the biasing function of an inserter instrument 300, described herein below. Thereafter, the inserter instrument 300 is drawn proximally within the lumen 102 of the retractor 100 until the locking tab 33 arm 34 of the extension clip 30 is compressed within the variably dimensioned slot 114 at its narrower locking slot 116. As shown in the depicted embodiment, the locking tab 23 and its tab arm 24 are compressed into engagement with opposing nubs 117 on the locking slot 116 which are biased into engagement with locking divots 32 in the tab arm 34.

It will be appreciated, of course, that other cooperating structures on each of the extension clip 30 and variably dimensioned slot 114 may be used for achieving locking. For example, the locking tab arm 34 may have a male tapered wedge shape and the locking slot 116 may be similarly shaped with a female wedge shaped such that the fixed engagement of a Morse-type taper is achieved. The tab arm 34 and locking slot 116 may include other features such as one or more of ribs, grooves, knurling and other surface texturing as alternative means for achieving releasable engagement or for enhancing other engagement means such as the tapers, and the depicted nubs 117 and locking divots 32. Removal of the extension clip 30 may be achieved by reversing the actions of the inserter instrument 300 which include engaging the insertion retainer 314 with the tool engagement aperture 35 by squeezing the handles, then extracting the extension clip 30 from the locked fit with the slot 104 by directing the instrument towards the distal end of the lumen 102 then opening the handles to decompress the insertion and biasing arms and thereafter remove the extension clip 30 from the lumen slot 104.

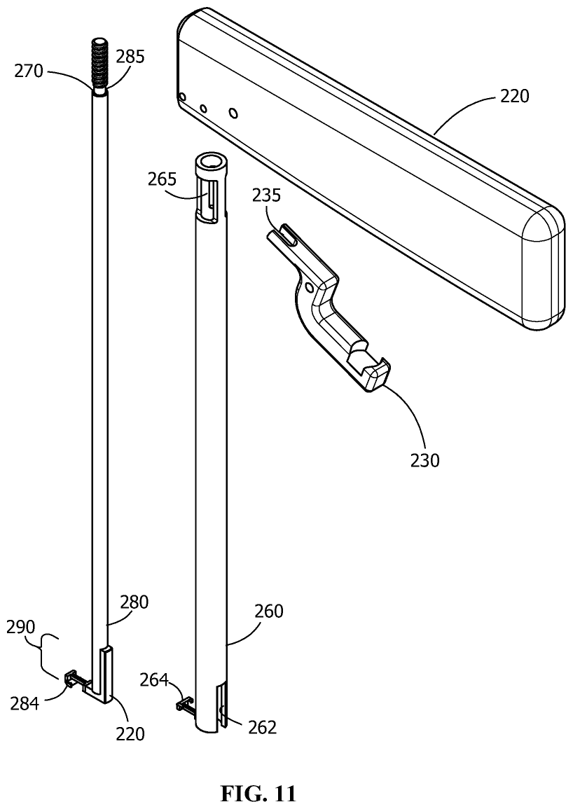

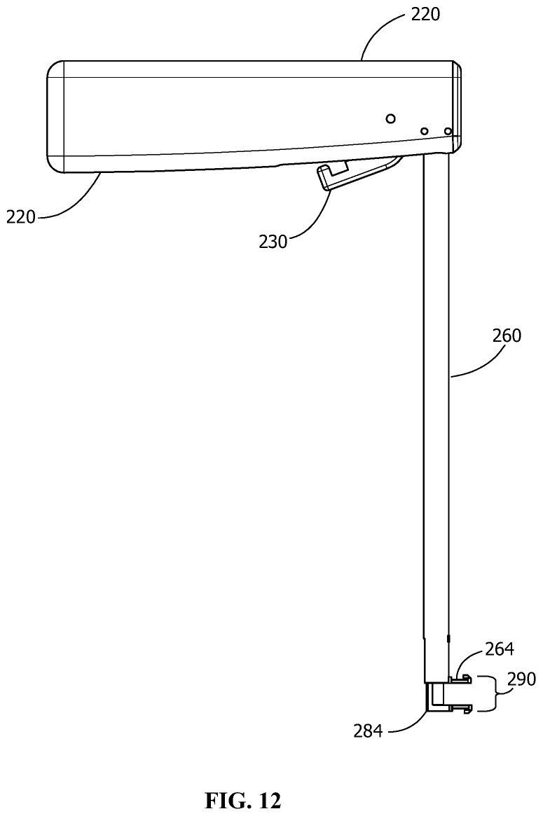

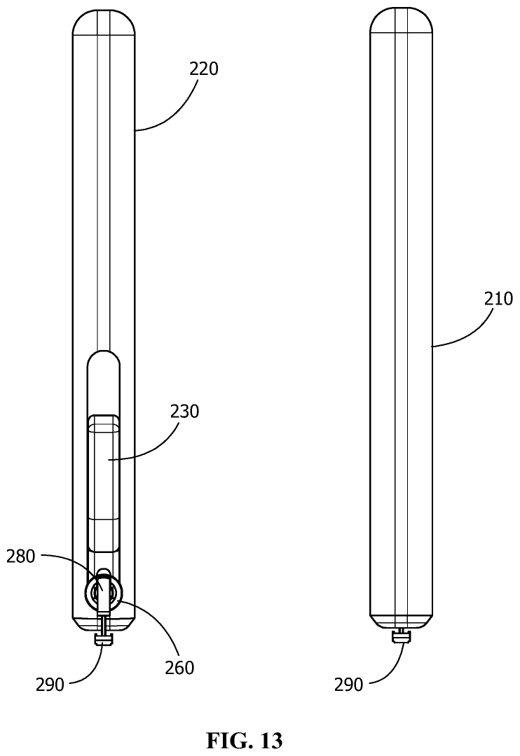

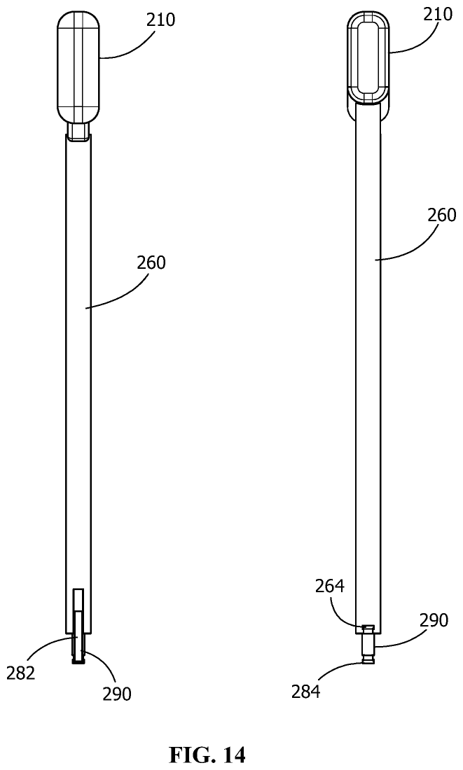

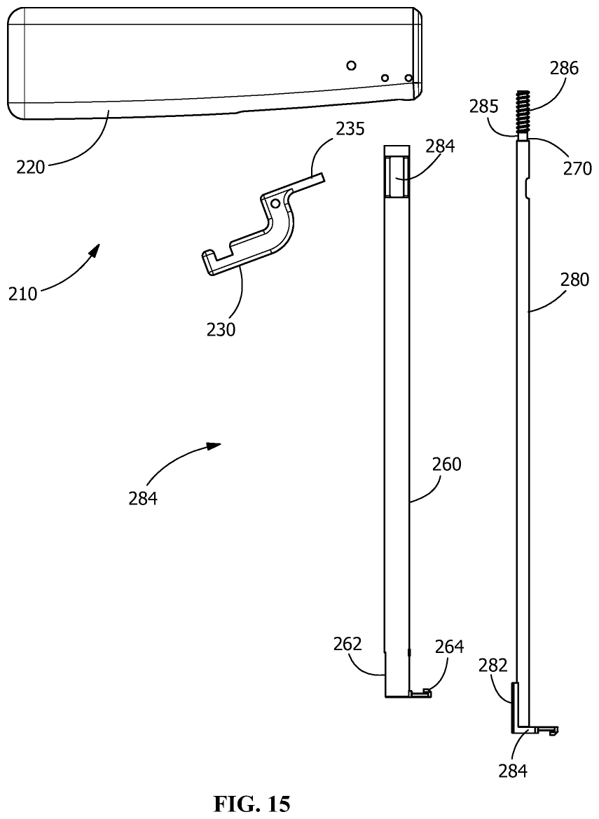

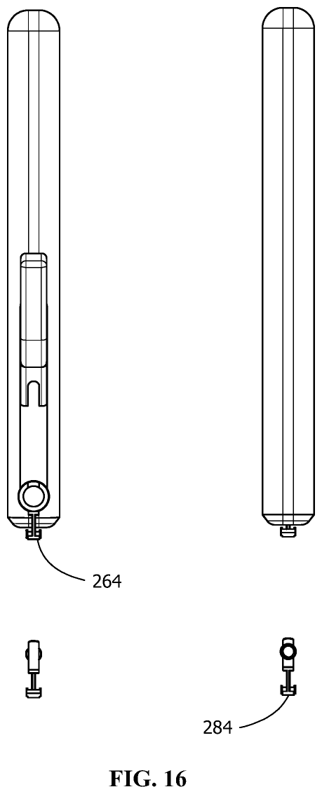

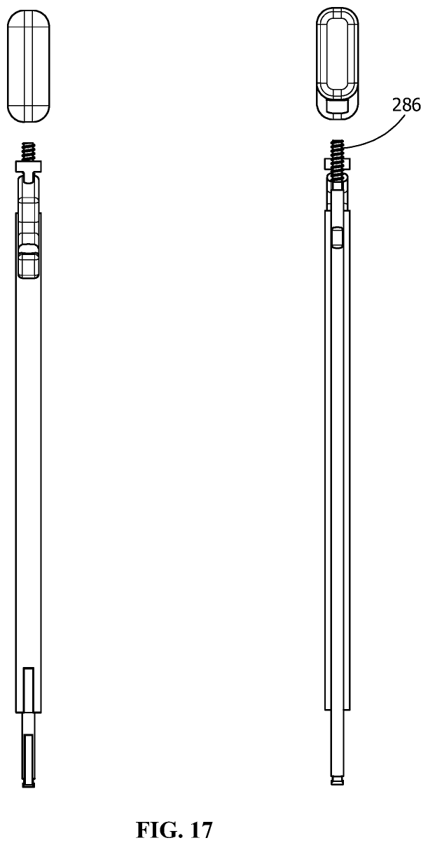

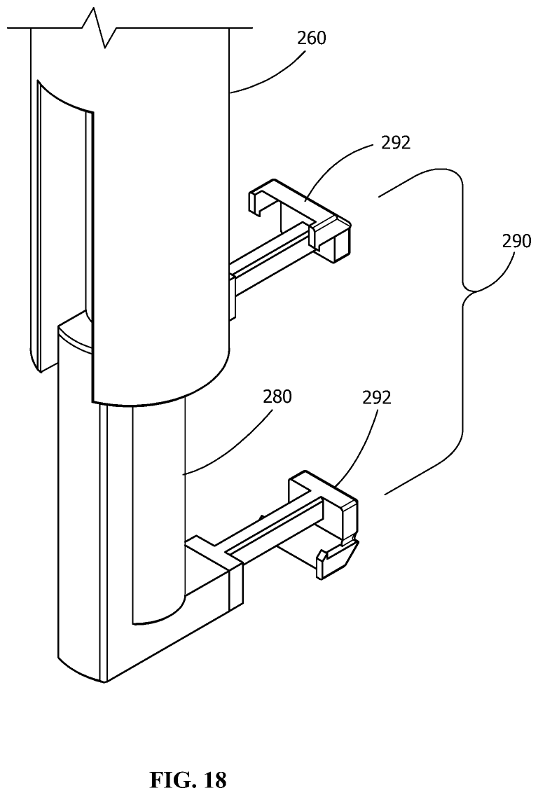

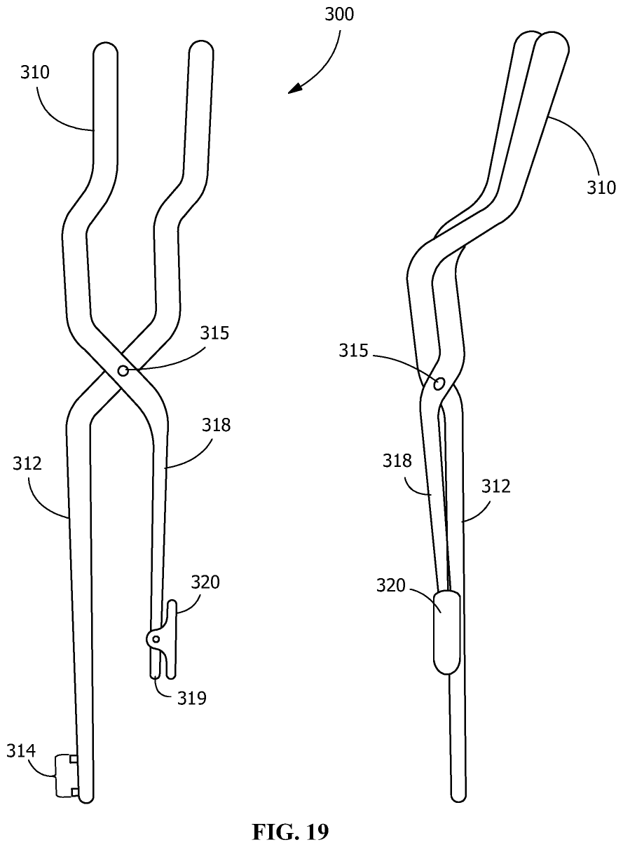

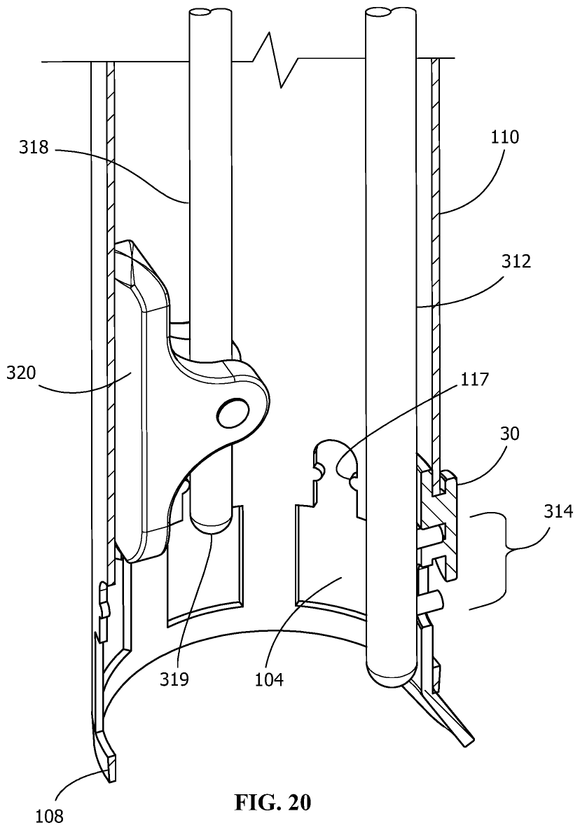

Referring now to the drawings at FIG. 10-FIG. 18, a first embodiment of a specialized deployment instrument 200 is provided that enables precise manipulation of an extension clip for placement and removal while the retractor is positioned in the surgical field within the patient. The deployment instrument 200 operates to facilitate engagement and disengagement of the extension mounting means with one or more slots of the retractor tube. Referring now to an exemplary extension clip 10 as shown in FIG. 1. In accordance with the depicted embodiment, the mounting means 11 is actuated by compression of the flexible tabs 12 toward one another, as described herein above. Thus, the deployment instrument 200 operates to contact each of the opposing hooks 13 and compresses them toward one another as a result of the flexion of the flexible tabs 12, either to enable insertion into a slot 104 or to enable extraction from within a slot 104.

As depicted, the opposing hooks 13 of the mounting means 11 are arranged for compression along the long axis of the extension clip 10, thus the deployment instrument 200 is adapted with opposing engagement grips 264, 284 that are each, respectively, adapted to at least partially capture each of the respective opposing hooks 13 and thereby manipulate the compression and release of the opposing hooks 13 for passage through a slot 104 in a retractor 100. The deployment instrument 200 includes an actuator handle 210, that includes a handle 220 and a trigger 230. The deployment instrument 200 also includes an actuator body 250, that includes an elongate cylindrical body housing 260 with a trigger guide 265 at its proximal end and a stabilizing reciprocating-grip retainer guide 262 at its distal end, the guides 262, 265 severing to stabilize the engagement and alignment of the engaged trigger 230 and actuator body 250. The actuator body 250 includes an elongate cylindrical reciprocating-grip body 280 that is oriented coaxially within the body housing 260 to which the fixed opposing grip 264 is mounted.

The components of the actuator body 250 cooperate by mechanical action along the shared elongate axis to provide a releasable gripper 290 that can grasp the extension clip 10 and mounting means 11. FIG. 18 shows in greater detail the features of the releasable gripper 290, which includes opposing clamps 292, each clamp respectively presented on each of the fixed opposing grip 264 and the reciprocating opposing grip 284. As shown, each opposing clamp 292 is configured to partially enclose the hook 13, to thereby control its motion from between an opened and a compressed configuration. It will be appreciated that in some other embodiments, the shape of the grip features may vary, and for example, may be boxed shaped, or hemispherical, or conical, the key aspect being configured to releasably capture the hook 13 for compression.

Referring again to the drawings, the actuator body 250 extends in a perpendicular orientation from the actuator handle 210 and provides a spring-loaded action via a spring 286 mounted on the proximal end of the reciprocating grip body 280. The trigger 230 is engaged with the spring loaded reciprocating grip body 280 at a proximally oriented trigger mount 285, that enables the reciprocal movement of the distally located reciprocating opposing grip 284 along the elongate axis. The adjacent fixed grip body housing 260 includes a fixed opposing grip 264 at its distal end. In use, the trigger actuates reciprocal movement of the reciprocating grip body 280 axially within the body housing 260.

According to the depicted configuration, at rest, the spring 286 is relaxed and the releasable gripper 290 is in the open configuration. When the trigger is actuated, the spring 286 can be adjustably compressed between the trigger flange 235 and the top of the body housing 260 to thereby draw the reciprocating grip body 280 proximally and bringing each of the opposing grips 264, 284 towards one another. The reciprocating grip retainer 282 is configured to interfere with the reciprocating grip retainer guide 262 to stop the compression of the spring 286. Upon release of the trigger 230, the spring 286 is relaxed to thereby open the releasable gripper 290. Thus, trigger 230 actuation moves the reciprocating opposing grip 284 towards or away from the fixed opposing grip 264.

It should be appreciated that in other embodiments, an alternate arrangement of the spring mechanism may be employed such that in the relaxed state of the spring, the releasable gripper 290 is held in a closed configuration, and compression of the spring 286 actuates its opening. In yet other embodiments, the system may be adapted to achieve actuation along an axis that is not parallel with the axis of the actuator body 250. For example, the mechanism may be adapted to actuate a distal gripper along an axis that is perpendicular to the long axis of the actuator body 250. In other alternative embodiments, cams or gears may be employed to provide rotational motion of the gripper.

For purposes of manipulating an extension clip 10 such as the one shown in FIG. 1, the trigger is actuated between open and closed configurations to thereby engage and compress the extension mounting means 11 of the extension clip 10 for insertion or removal within a retractor slot 104. Reverse actuation of the trigger 230 releases the engagement with the mounting means. Thus, in operation, the mounting means 11 is compressed by engagement with the deployment instrument 200 whereby the mounting means 11 is inserted into a slot 104, and upon disengagement of the deployment instrument 200, the mounting means 11 is fixed within the slot 104. Reengagement of the deployment instrument 200 with the mounting means 11 enables disengagement of the extension clip 10 from the slot 104.

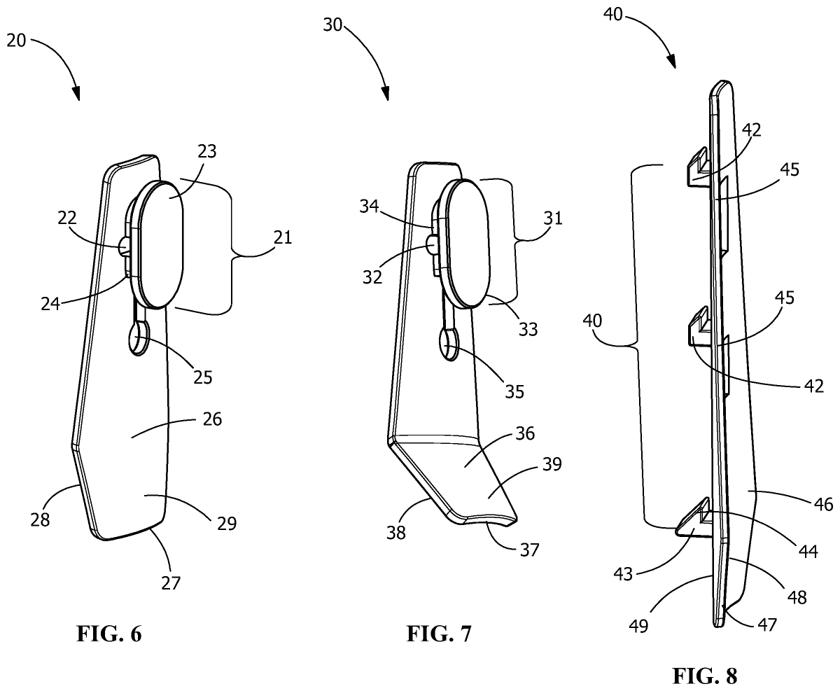

Referring now to FIG. 19, an alternate embodiment of an inserter instrument 300 is shown, the instrument being particularly suitable for use with extension clips having mounting means that do not rely on compression of opposing hooks or similar engagement features such as those present in the extension clip 10 shown in FIG. 1 and FIG. 4. In some examples, the inserter instrument 300 depicted in FIG. 19 may be used with extension clips such as the embodiments shown in each of FIG. 2, FIG. 3, FIG. 4, FIG. 6, FIG. 7 and FIG. 8. Referring now to FIG. 20, in a particular example, the extension clip 30, which is further detailed in FIG. 7, is shown engaged with a retractor tubular body 110.

Referring again to FIG. 20, the depicted inserter instrument 300 operates by guiding and then compressing the extension clip into engagement with the retractor slot by snap or compression fitting. The inserter instrument 300 includes proximally oriented opposing scissor like handles 310 and opposing arms that include an insertion arm 312 and a biasing arm 318, the handles 310 and the insertion arm 312 and biasing arm 318 being joined by a hinge 315 positioned therebetween. The biasing arm 318 includes at a distal biasing means 319 that is adapted to be compressed against the internal wall of the retractor 100 lumen 102 when opposite the position of a slot 104 into which an extension clip is to be inserted, the compression actuated by squeezing the handles 310.

Referring again to FIG. 20, the depicted extension clip 30 is adapted with a mounting means 31 that includes a locking tab 33 affixed by a tab arm 34 having one each on opposing sides of the tab arm 34 a locking divot 32. In use, the distal biasing means 319 may include a torque plate or other planar or curved feature to enhance the surface area of contact with the lumen 102 wall. In some instance, the plate may fit or engage with one or more slots. As shown in FIG. 19, the pivoting counter torque plate 320 has a disc shape and is mounted to the biasing arm 318 via a plate hinge 321. This feature not only enhances the surface contact with the lumen 102 to stabilize the instrument for guiding insertion of the extension clip, it also provides enhanced maneuverability to allow for finer motion of the instrument to aid insertion and engagement of an extension clip that is affixed to the insertion arm 312. Referring again to FIG. 19, specifically in reference to the insertion arm 312, an insertion retainer 314 feature is depicted. The insertion retainer 314 is adapted to engage with an extension clip via one or more engagement apertures to removably engage the extension clip with the inserter instrument 300 for fixed placement in a retractor 100 slot 104.

Referring again to FIG. 20, the inserter instrument 300 in use may be manipulated to align and affix a variety of extension clips. In one exemplary embodiment wherein the inserter instrument 300 may be used, the extension clip 30 as shown in FIG. 7 may be releasably engageable within a variably dimensioned slot 114 by compression locking engagement. In one such embodiment, a retractor adapted with at least one variably dimensioned slot 114, as shown in the depicted embodiment, wherein a locking tab 33 of an extension clip 30 may be inserted into the variably dimensioned slot 114 at its wider insertion slot 115, aided by the biasing function of the inserter instrument 300. Thereafter, the inserter instrument 300 is drawn proximally within the lumen 102 of the retractor 100 until the locking tab arm 34 of the extension clip 30 is compressed within the variably dimensioned slot 114 at its narrower locking slot 116. As shown in the depicted embodiment, the locking tab 33 and its tab arm 34 are compressed into engagement with opposing nubs 117 on the locking slot 116 which are biased into engagement with locking divots 32 in the tab arm 34.

In yet another exemplary embodiment wherein the inserter instrument 300 may be used, the extension clip 40, as shown in FIG. 8, may be releasably engageable by a snap fit locking engagement. In one such embodiment, a retractor having at least two vertically adjacent slots 104, is used, wherein a mounting means 41 that includes two vertically separated hooks 42, 43 and a locking flange 44 is engaged by snap fitting engagement to the distal end 108 of the retractor 100. In use, the inserter instrument 300 as described above is engageable with the extension clip 40 via its tool engagement aperture 45, and the slot hook 42 and distal hook 43 are directed into engagement with vertically separated slots 104; the inserter instrument 300 is then rotated within the lumen 102 in a distal direction until the distal locking flange 44 snaps on to the distal end 108 of the tubular body 110 of the retractor 100. The process is reversed for removal of the clip, wherein a grasping member affixed to the distal end of the insertion arm is directed to grasp the distal locking flange 44 of the clip 40 to unsnap its fixation with the retractor 100.

As is described herein above, a key aspect of the engagement features of the instant invention is that the forces required to unlock the engagement features of the extension clip and slot must be directed towards the distal end of the retractor tube, whereas the direction of the forces against the extension clips are either lateral or upward. In use, the counter forces on the retractor tips come primarily from (i) lateral forces directed against the front of the distal blades due to contact with surgical tools, (ii) lateral forces against the back of the distal blades due to pressure exerted by the soft tissue, and (iii) proximally directed vertical forces due to pressure exerted by the soft tissue. The direction of these forces would not operate to disengage the locked clips.

It is contemplated that the invention may be provided to surgeons or other users in the form of a kit, such kit comprising a retractor having at least one slot and a plurality of extension clips of varying shapes and sizes, a deployment instrument, and other optional components.

In some embodiments, extension clips may be deployed partially or wholly within the retractor and may be used for retention of one or more insertable instruments. In some examples these would include irrigation, suction, electro/neuromonitoring, fiber optic lighting, camera or other instruments to facilitate the surgical procedure. Thus, in addition to extension clips for clearance of soft tissue from a retractor, also provided herein are adaptations for retractor tubes to facilitate the securement of instruments within the tube. In some embodiments, the adaptations include one or more flexible clips that are adapted to be secured to the walls of the tube specifically for retaining in place one or more instruments. In use, the instrument, such as a tubular light, camera, neuro-monitoring cable, or the like is inserted in the tube into the surgical field and is clipped to the tube by engagement of the adapted extension clips within in the slots.

In use, an array of the extension clips is provided with retractors adapted for engagement with at least one more of the array of extension clips. Upon accessing the surgical field in a patient, a retractor and clip combination are selected, and the retractor is inserted into position in the field according to standard protocol. Thereafter, the one or more suitably dimensioned extension clips are selected, and serially, each is engaged with the appropriate insertion instrument. The engaged instrument and clip are passed into the lumen of the retractor body. As determined by the surgeon, the distal end of the clip may be maneuvered first into contact with the soft tissue below/adjacent the distal end of the retractor so as to initially displace the tissue away from the distal lumen opening. While retaining contact with the displaced soft tissue, the surgeon extension clip mounting means is guided into initial alignment with the retractor slot/slots and the tool is thereafter manipulated to actuate insertion and engagement of the mounting means with the retractor slot/slots. Multiple extension clips may be deployed, including clips with varied configurations as selected by the surgeon to address the anatomical variations within the soft tissue adjacent the retractor. After engagement and locking is achieved, the insertion tool is disengaged and withdrawn from the retractor lumen. In some instances, the tool may be reinserted into the lumen to adjust the position of or remove the extension clip. In some examples the instrument may be used with another instrument to achieve unlocking of the extension clip prior to extraction from the retractor slot. Upon completion of the surgical procedure, one or more of the extension clips may be detached and removed prior to removal of the retractor, or they may be left in place and removed with the retractor member.

Embodiments of the present invention are suitable for use, in some examples, in a posterior or transforaminal approach for spinal surgery, and may be adapted for uses in other spinal surgical orientations and other surgical sites within the body.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

The general inventive concepts may be embodied in different forms and should not be construed as limited to the embodiments set forth herein. Rather, these embodiments are provided so that this disclosure will be thorough and complete, and will fully convey the scope of the general inventive concepts to those skilled in the art.

As used herein, the singular forms “a,” “an,” and “the” are intended to include the plural forms as well, unless the context clearly indicates otherwise. The term “proximal” as used in connection with any object refers to the portion of the object that is closest to the operator of the object (or some other stated reference point), and the term “distal” refers to the portion of the object that is farthest from the operator of the object (or some other stated reference point). The term “operator” means and refers to any professional or paraprofessional who delivers clinical care to a medical patient, particularly in connection with the delivery of care.

Anatomical references as used herein are intended to have the standard meaning for such terms as understood in the medical community. For example, the application may include reference to the following terms: “cephalad,” “cranial” and “superior” indicate a direction toward the head, and the terms “caudad” and “inferior” indicate a direction toward the feet. Likewise, the terms “dorsal” and “posterior” indicate a direction toward the back, and the terms “ventral” and “anterior” indicate a direction toward the front. And the term “lateral” indicates a direction toward a side of the patient. The term “medial” indicates a direction toward the mid line of the patient, and away from the side, the term “ipsilateral” indicates a direction toward a side that is proximal to the operator or the object being referenced, and the term “contralateral” indicates a direction toward a side that is distal to the operator or the object being referenced. And, more specifically with respect to the directional movement of an implant according to the methods of the disclosure, sideways refers to the general direction of movement within the disc space between the endplates from the position of the inserted instruments toward one or the other of the contralateral and ipsilateral portions of the disc space. In the case of a TLIF procedure, such sideways motion will generally be in a medial direction relative to the disc space. Though in other types of surgical access, particularly within the spine, sideways movement may be either medial or lateral relative to the disc space, and in other surgical contexts sideways is away from the initial position of the implant. Further, with respect to the movement of an implant by action of the surgical instruments, the movement may also be rotational, wherein the action of the instruments directs the implant sideways and also in a rotational or pivotal motion. More generally, any and all terms providing spatial references to anatomical features shall have meaning that is customary in the art.

Unless otherwise indicated, all numbers expressing quantities, properties, and so forth as used in the specification, drawings and claims are to be understood as being modified in all instances by the term “about.” Accordingly, unless otherwise indicated, the numerical properties set forth in the specification and claims are approximations that may vary depending on the suitable properties desired in embodiments of the present invention. Notwithstanding that the numerical ranges and parameters setting forth the broad scope of the general inventive concepts are approximations, the numerical values set forth in the specific examples are reported as precisely as possible. Any numerical values, however, inherently contain certain errors necessarily resulting from error found in their respective measurements.

References to visualization using radiography as may be described in the exemplary techniques herein are merely representative of the options for the operator to visualize the surgical field and the patient in one of many available modalities. It will be understood by one of ordinary skill in the art that alternate devices and alternate modalities of visualization may be employed depending on the availability in the operating room, the preferences of the operator and other factors relating to exposure limits. While confirmation of instrument placement in the course of the technique is appropriate, the frequency and timing relative to the sequence of steps in the technique may be varied and the description herein is not intended to be limiting. Accordingly, more or fewer images, from more or fewer perspectives, may be collected.

One of ordinary skill will appreciate that references to positions in the body are merely representative for a particular surgical approach. Further, all references herein are made in the context of the representative images shown in the drawings. Fewer or additional instruments, including generic instruments, may be used according to the preference of the operator. Moreover, references herein to specific instruments are not intended to be limiting in terms of the options for use of other instruments where generic options are available, or according to the preference of the operator.

While the disclosed embodiments have been described and depicted in the drawings in the context of the human spine, it should be understood by one of ordinary skill that all or various aspects of the embodiments hereof may be used in connection with other species and within any species on other parts of the body where deep access within the tissue is desirable.

While various inventive aspects, concepts and features of the general inventive concepts are described and illustrated herein in the context of various exemplary embodiments, these various aspects, concepts and features may be used in many alternative embodiments, either individually or in various combinations and sub-combinations thereof. Unless expressly excluded herein all such combinations and sub-combinations are intended to be within the scope of the general inventive concepts. Still further, while various alternative embodiments as to the various aspects, concepts and features of the inventions (such as alternative materials, structures, configurations, methods, devices and components, alternatives as to form, fit and function, and so on) may be described herein, such descriptions are not intended to be a complete or exhaustive list of available alternative embodiments, whether presently known or later developed.

Those skilled in the art may readily adopt one or more of the inventive aspects, concepts and features into additional embodiments and uses within the scope of the general inventive concepts, even if such embodiments are not expressly disclosed herein. Additionally, even though some features, concepts and aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated.

Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated.

What is claimed is:

1. A surgical retractor system comprising:
(i) a surgical retractor having an elongate body bounded by a proximal end and a distal end, and at least one slot positioned through a wall of the body at a point between the proximal and the distal ends,
(ii) an extension clip having a flared blade, and a mounting means for releasable engagement with the at least one slot of the surgical retractor, the mounting means comprising one or more of a spring type fitting that is actuatable between open and compressed configurations, an interference fitting, a compression fitting, and a snap fitting; and
(iii) a deployment instrument comprising
an instrument body with a releasable actuation means for engaging the mounting means of the extension clip and for one of affixing or releasing the extension clip in relation to the surgical retractor slot, the actuation means adapted to direct locking engagement of the extension clip mounting means with the surgical retractor, the actuation means comprising one or more of a releasable gripper for grasping and compressing a spring type fitting, and a biasing means for effecting one or more of compression and snap fitting engagement; and
an actuator handle in operable communication with the releasable actuation means of the instrument body
wherein the system is adapted for engagement of the extension clip with the surgical retractor body slot to mount the extension in a locked position for extension of the blade toward or outside of the distal end of the surgical retractor body,
whereby, when fixed to the surgical retractor, at least a portion of the extension blade is positioned to contact soft tissue adjacent the distal end of the surgical retractor and thereby minimize tissue creep into the surgical retractor.

2. A surgical retractor system according to claim 1, the mounting means of the extension clip comprising a spring-type interference fitting that is adapted to be actuated between open and compressed configurations, wherein in the compressed configuration, the mounting means can be passed through the surgical retractor slot, and in the open configuration the mounting means is retained in the surgical retractor slot by interference fitting, the extension clip mounting means comprising opposing flexible tabs that are proximal to the distal blade, each tab comprising a hook that includes a pull out stop for interference with the surgical retractor wall when inserted in the slot, the tabs being separated from each other by an open aperture, the mounting means being actuatable by flexion of the tabs within the aperture to move the hooks toward each other in a compressed configuration and released to return to an open configuration, the actuation allowing for passage of the hooks through the slot; and
wherein the actuation means of the deployment instrument includes a releasable gripper comprising opposing grips that are engageable with the extension clip mounting means, the handle comprising a trigger that alternately directs the action of the opposing grips between open and closed configurations, whereby the releasable gripper actuates the mounting means between open and closed configurations to facilitate passage of the mounting means hooks through the surgical retractor slot.

3. A surgical retractor system according to claim 2, wherein the gripper operates by reciprocation of at least one of the opposing grips, the reciprocation being along one of an axis defined along the surgical retractor body between its proximal and distal ends, and an axis that is transverse to the axis along the surgical retractor body between its proximal and distal ends.

4. A surgical retractor system according to claim 2, the surgical retractor comprising a generally cylindrical or ellipsoid elongate body defining a lumen between its proximal and distal ends and comprising at least one slot disposed through the elongate body toward the distal end.

5. A surgical retractor system according to claim 1, the mounting means of the extension clip comprising a locking tab affixed by a tab arm and extending from a back side of the extension clip, the tab arm comprising locking divots on opposing sides of the tab arm, and
the surgical retractor comprising a generally cylindrical or ellipsoid elongate body defining a lumen between its proximal and distal ends and comprising at least one slot disposed through the elongate body toward the distal end, the slot being variably dimensioned along a length between the proximal and distal ends of the surgical retractor, the variably dimensioned slot comprising a distally oriented portion and a proximally oriented portion, the proximally oriented portion having a width dimension that is smaller than a width dimension of the distally oriented portion and the proximally oriented portion comprising on each of opposing internal side edges of the wall one or more nubs adapted for interfitting with the locking divots on the locking tab arm of the extension clip; and
wherein the deployment instrument operates by guiding and then directing the locking tab of the extension clip into engagement with the distal portion of the slot, then guiding the extension clip proximally until the arm of the locking tab engages with the proximal portion of the slot, whereby the extension clip is releasably locked to the surgical retractor by engagement between the locking divots and nubs, and is prevented from pull out by interference between the locking tab and the surgical retractor body.

6. A surgical retractor system according to claim 5, the deployment instrument comprising scissor like handles at a proximal end, and oriented distally, opposing arms that include an insertion arm and a biasing arm, the handles and the insertion and biasing arms being joined by a hinge positioned therebetween, the biasing arm further comprising a distal biasing means that is adapted to be compressed against the surgical retractor wall when opposite the slot, whereby engagement between the mounting means of the extension clip and the slot being initially directed by squeezing the handles.

7. A surgical retractor system according to claim 1, the mounting means of the extension clip comprising a locking tab affixed by a tab arm and extending from a back side of the extension clip, the tab arm having a cross sectional shape from distal to proximal that is conical or frustoconical, and
the surgical retractor comprising a generally cylindrical or ellipsoid elongate body defining a lumen between its proximal and distal ends and comprising at least one slot disposed through the elongate body toward the distal end, the slot being variably dimensioned along a length between the proximal and distal ends of the surgical retractor, the variably dimensioned slot comprising a distally oriented portion and a proximally oriented portion, the distally oriented portion having a greater width dimension than a width dimension of the proximally oriented portion, the proximally oriented portion tapering from the distally oriented portion in a shape that is conical or frustoconical;
wherein the deployment instrument operates by guiding and then directing the locking tab of the extension clip into engagement with the distal portion of the slot, then guiding the extension clip proximally until the arm of the locking tab engages with the proximal portion of the slot into frictional engagement, whereby the extension clip is releasably locked to the surgical retractor by engagement between the tapered portions of the slot and the locking tab arm, and is prevented from pull out by interference between the locking tab and the surgical retractor body.

8. A surgical retractor system according to claim 7, the deployment instrument comprising scissor like handles at a proximal end, and oriented distally, opposing arms that include an insertion arm and a biasing arm, the handles and the insertion and biasing arms being joined by a hinge positioned therebetween, the biasing arm further comprising a distal biasing means that is adapted to be compressed against the surgical retractor wall when opposite the slot, whereby engagement between the mounting means of the extension clip and the slot being initially directed by squeezing the handles.

9. A surgical retractor system according to claim 1, the mounting means of the extension clip comprising at least two vertically separated hooks directed proximally and extending from a back side of the extension clip, wherein at least one of the hooks comprises a locking flange that is directed proximally and is positioned distally on the extension clip,
the surgical retractor comprising a generally cylindrical or ellipsoid elongate body defining a lumen between its proximal and distal ends and comprising one of at least one slot and two or more slots that are vertically separated, the one or more slots disposed through the elongate body and positioned toward the distal end,
wherein the deployment instrument operates by guiding and then directing at least one of the at least two vertically separated hooks into engagement with at least one slot, whereby snap fit locking of the distal locking flange to one of a slot or the wall of the surgical retractor at its distal end is achieved by actuating the deployment instrument to rotate the extension clip within the lumen in a distal direction until the distal locking flange contacts and snaps on to the slot or the distal end of the tubular body of the surgical retractor.

10. A surgical retractor system according to claim 1, the mounting means of the extension clip comprising at least two vertically separated hooks directed proximally and extending from a back side of the extension clip, and at least one distally positioned locking flange directed proximally,
the surgical retractor comprising a generally cylindrical or ellipsoid elongate body defining a lumen between its proximal and distal ends and comprising at least two vertically adjacent slots disposed through the elongate body and positioned toward the distal end,
wherein the deployment instrument operates by guiding and then directing the at least two vertically separated hooks into engagement, respectively, with the at least two vertically separated slots, whereby snap fit locking of the distal locking flange to the wall of the surgical retractor at its distal end is achieved by actuating the deployment instrument to rotate the extension clip within the lumen in a distal direction until the distal locking flange contacts and snaps on to the distal end of the tubular body of the surgical retractor.

11. An extension clip for a surgical retractor comprising:
a body comprising at a proximal end a mounting means for releasable engagement with at least one slot of the surgical retractor, the mounting means comprising one or more of a spring type fitting that is actuatable between open and compressed configurations, an interference fitting, a compression fitting, and a snap fitting, and at a distal end a flared blade
wherein the extension clip is adapted for engagement with the surgical retractor slot in a releasably locked position for positioning the blade toward or outside of a distal end of the surgical retractor,
whereby, when fixed to the surgical retractor, at least a portion of the extension blade is positioned to contact soft tissue adjacent the distal end of the surgical retractor and thereby minimize tissue creep into the surgical retractor.

12. An extension clip according to claim 11, the mounting means of the extension clip comprising a spring-type interference fitting that is adapted to be actuated between open and compressed configurations, wherein in the compressed configuration, the mounting means can be passed through the surgical retractor slot, and in the open configuration the mounting means is retained in the surgical retractor slot by interference fitting, the extension clip mounting means comprising opposing flexible tabs that are proximal to the distal blade, each tab comprising a hook that includes a pull out stop for interference with a wall of the surgical retractor when inserted in the slot, the tabs being separated from each other by an open aperture, the mounting means being actuatable by flexion of the tabs within the aperture to move the hooks toward each other in a compressed configuration and released to return to an open configuration, the actuation allowing for passage of the hooks through the slot; and wherein the mounting means is actuatable between open and closed configurations to facilitate passage of the mounting means hooks through the surgical retractor slot.

13. An extension clip according to claim 11, the mounting means of the extension clip comprising a locking tab affixed by a tab arm and extending from a back side of the extension clip, the tab arm comprising locking divots on opposing sides of the tab arm,
wherein the arm of the locking tab is engageable with a surgical retractor slot that is variably dimensioned along a length between proximal and distal ends of the surgical retractor, the variably dimensioned slot comprising a distally oriented portion having a width dimension that is greater than a width dimension of a proximally oriented portion,
whereby upon its passage into the distally oriented slot portion and proximal translation into the proximally oriented slot portion, the extension clip is releasably locked to the surgical retractor, and is prevented from pull out by interference between the locking tab and the surgical retractor body.

14. An extension clip according to claim 11, the mounting means of the extension clip comprising a locking tab affixed by a tab arm and extending from a back side of the extension clip, the tab arm having a cross sectional shape from distal to proximal that is conical or frustoconical, wherein the arm of the locking tab is engageable with a surgical retractor slot that is variably dimensioned along a length between the proximal and distal ends of the surgical retractor, the variably dimensioned slot comprising a distally oriented portion and a proximally oriented portion, the distally oriented portion having a greater width dimension than a width dimension of the proximally oriented portion, the proximally oriented portion tapering from the distally oriented portion in a shape that is conical or frustoconical;
whereby upon its passage into the distally oriented slot portion and proximal translation into the proximally oriented slot portion, the extension clip is releasably locked to the surgical retractor, and is prevented from pull out by interference between the locking tab and the surgical retractor body.

15. An extension clip according to claim 11, the mounting means of the extension clip comprising at least two vertically separated hooks directed proximally and extending from a back side of the extension clip, and at least one distally positioned locking flange directed proximally,
wherein the at least two vertically separated hooks are engagable, respectively, with the at least two vertically separated slots in the surgical retractor,
whereby the distal locking flange is releasably locked to a distally positioned edge of the surgical retractor achieved by actuating snap fit locking.

16. A surgical retractor extension deployment instrument, comprising
an instrument body with a releasable actuation means for engaging a mounting means of the extension clip and for one of affixing or releasing the extension clip in relation to at least one slot in the surgical retractor, the actuation means adapted to direct locking engagement of the extension clip mounting means with the surgical retractor, the actuation means comprising one or more of a releasable gripper for grasping and compressing a spring type fitting, and a biasing means for effecting one or more of interference, compression and snap fitting engagement; and
an actuator handle in operable communication with the releasable actuation means of the instrument body,
wherein the instrument is adapted for engagement of the extension clip with the surgical retractor slot to mount the extension clip in a locked position for positioning the blade toward or outside of a distal end of the surgical retractor body,
whereby, when fixed to the surgical retractor, at least a portion of the extension blade is positioned to contact soft tissue adjacent the distal end of the surgical retractor and thereby minimize tissue creep into the surgical retractor.

17. The surgical retractor extension deployment instrument according to claim 16, wherein the actuation means of the deployment instrument includes a releasable gripper comprising opposing grips that are engageable with the extension clip mounting means, the handle comprising a trigger that alternately directs the action of the opposing grips between open and closed configurations.

18. The surgical retractor extension deployment instrument according to claim 16, the deployment instrument comprising scissor like handles at a proximal end, and oriented distally, opposing arms that include an insertion arm and a biasing arm, the handles and the insertion and biasing arms being joined by a hinge positioned therebetween, the biasing arm further comprising a distal biasing means that is adapted to be compressed against a surface that opposes a slot that is adapted to releasably receive an extension clip when the handles are one of squeezed and released, wherein the biasing means optionally includes on the biasing arm a torque plate or other planar or curved feature to enhance the contact surface area of the biasing arm.

19. The surgical retractor extension deployment instrument according to claim 18, the instrument adapted for use with a surgical retractor comprising a generally cylindrical or ellipsoid elongate body defining a lumen between its proximal and distal ends and comprising at least one slot disposed through the elongate body toward the distal end, the slot being variably dimensioned along a length between the proximal and distal ends of the surgical retractor, the variably dimensioned slot comprising a distally oriented portion and a proximally oriented portion, the proximally oriented portion having a width dimension that is smaller than a width dimension of the distally oriented portion and the proximally oriented portion comprising on each of opposing internal side edges of the wall one or more nubs adapted for interfitting with the locking divots on the locking tab arm of the extension clip, wherein the deployment instrument operates by guiding and then directing a locking tab of the extension clip into engagement with the distal portion of the slot, then guiding the extension clip proximally until the arm of the locking tab engages with the proximal portion of the slot, whereby the extension clip is releasably locked to the surgical retractor by engagement between the locking divots and nubs, and is prevented from pull out by interference between the locking tab and the surgical retractor body.

20. The surgical retractor extension deployment instrument according to claim 18, the instrument adapted for use with a surgical retractor comprising at least two vertically adjacent slots, wherein the deployment instrument operates by guiding and then directing at least two vertically separated hooks on the extension clip into engagement, respectively, with the at least two vertically separated slots, whereby snap fit locking of a distal locking flange on the extension clip to the surgical retractor at a distal edge is achieved by actuating the deployment instrument to rotate the extension clip until the distal locking flange contacts and snaps on to the distal edge of the surgical retractor.
